(19) United States
(12) Patent Application Publication
(10) Pub. No.: US 2006/0177916 A1
(43) Pub. Date: Aug. 10, 2006

(54) Ethanol production from citrus processing waste

(75) Inventors: David A. Stewart, Boca Raton, FL (US); Wilbur W. Widmer, Winter Haven, FL (US); Karel Grohmann, Davenport, FL (US); Mark R. Wilkins, Winter Haven, FL (US)

Correspondence Address: EDWARD M. LIVINGSTON, PA, 963 TRAIL TERRACE DRIVE, NAPLES, FL, 34103, US

(73) Assignees: Renewable Spirits, LLC; The U.S. Department of Agriculture

(21) Appl. No.: 11/052,620

(22) Filed: Feb. 7, 2005

(51) Int. Cl. C12P 7/06

(52) U.S. Cl. 435/161

(57) ABSTRACT

Processes for producing ethanol from citrus waste by reducing the concentration of limonene in citrus waste to allow fermentation are disclosed. In one embodiment a slurry of ground citrus waste 1 is partially hydrolyzed by heating using a jet cooker 2 and then injected into a flash tank 4 to remove limonene 5. The heated citrus waste is then cooled, hydrolyzed with enzymes and fermented to ethanol. An alternative method of limonene removal uses enzymatic hydrolysis followed by centrifugation 27 to separate sugar-containing liquid from residual citrus waste solids containing limonene. Sugars are fermented and ethanol is distilled from the fermented mixture/beer. The remaining solids and liquids may be processed further to yield other byproducts. More particularly, the solids may be dried and pressed for use in cattle feed and the liquids may be further fermented or processed to yield additional ethanol, acetate, galacturonic acid monomers and polymers, five carbon sugars and other products.

### BACKGROUND OF THE INVENTION

This invention relates to citrus waste processing and, more particularly, a process for the conversion of simple and complex carbohydrates contained in citrus waste into ethanol for use as bio-fuel and to yield other high-value byproducts.

Currently, the amount of citrus waste, consisting primarily of peel, membranes, and seeds, which result from processing citrus fruit for juice, is an environmental problem. The problem exists particularly in areas where the bulk of citrus is grown to produce juice, such as in the State of Florida and the country of Brazil. For example, in 2003 Florida had approximately 103 million citrus trees on 800,000 acres and produced 297 million boxes of citrus, 85% of which was processed into juice. The waste from such processing was approximately one-half of the citrus fruit, yielding approximately 5 million tons of wet waste, which reduces to 1.2 million tons of dry waste.

Traditionally, such waste has been converted into cattle feed, which currently does not have sufficient value to cover the production and transportation costs associated therewith. A further drawback of converting current waste into cattle feed is that the waste contains a high amount of d(+)-limonene (referred to simply as limonene). Volatilization of the limonene during the drying process causes air pollution to the extent that limonene vapors are exhausted into the atmosphere at the processing plants because it would require very expensive equipment to trap the limonene from the drier exhaust. Although citrus waste materials do create an environmental problem, these materials are rich in pectin and other polysaccharides that can be hydrolyzed into sugars for use in the production of ethanol.

Currently ethanol is used as a bio-fuel that is mixed with gasoline to increase the octane rating and improve the environmental characteristics of gasoline. Although another gasoline octane enhancer referred to as MTBE (Methyl Tertiary Butyl Ether) is also used, MTBE is controversial since it is believed to result in ground water pollution and is not biodegradable. Field corn (maize) is currently the primary feedstock for ethanol production in the USA. As the State of Florida has no cultivation of field corn, Florida must look to other sources for producing ethanol. The conversion of citrus processing waste into ethanol would reduce waste and provide a regional source of ethanol as a viable alternative octane enhancer to MTBE. The conversion of citrus processing waste in 2003 of approximately 5 million tons could result in potentially 100 million gallons of ethanol.

Unfortunately, one of the major problems that prevents processing citrus waste into ethanol is limonene. Limonene is a terpene-based liquid that is contained in citrus peel. Limonene provides a natural defense for the fruit against bacteria, viruses, molds, and other organisms. Accordingly, limonene protects the citrus waste from microbial buildup and fermentation by normal processes that would yield ethanol. It is also desirable to recover the limonene as a high value co-product. For efficient fermentation, limonene in the citrus waste must be reduced to a level below 3000 parts per million (preferred level below 1500 ppm). Thus, a need exists for processes that will decrease the amount of limonene in citrus processing waste in order to produce ethanol for use as a bio-fuel and other high value products, including cattle feed, limonene, five carbon sugars and galacturonic acid monomers and polymers.

The new processes disclosed herein, for processing citrus waste to ethanol, utilize enzyme mixtures of pectinase, hemicellulases, cellulases and beta-glucosidases for efficient hydrolysis of the complex carbohydrates in citrus waste residue into simple sugars. Two different processes, steam stripping or centrifuging, can be used to lower the limonene content in the citrus waste to a sufficiently low level whereby fermentation of the waste can efficiently produce ethanol. The fermentation utilizes traditional ethanol producing yeast, E. coli strain KO11, or other bacteria or fungi, followed by distillation to recover ethanol. The solids residue remaining may still be utilized as a cattle feed product and will have higher protein content than the citrus-based cattle feed currently being produced. The residue after distillation may also be pressed and filtered with optional recovery of acetate, five carbon sugars, or galacturonic acid monomers/polymers from the filtrate. Both jet cooking and centrifugation processes work more efficiently if the raw citrus processing waste is ground to a particle size of less than one inch (preferably less than one-half inch) using a hammer mill, grinding pump or similar shredding/chopping/grinding apparatus capable of handling and reducing said waste to the required size. A progressing cavity pump or similar pump (or conveyor) capable of pumping/moving raw or ground peel slurries with dry solids content up to thirty-five percent is then used to feed and mix the high viscosity mixture during the enzymatic hydrolysis and fermentation.

Once the particle size of solids in the raw citrus waste is reduced to a size sufficient for further processing, then in a preferred processing embodiment the ground peel is first heated to a range of 60°-240° Celsius (preferred range 90°-190° C.) by steam injection, passage through a heated hollow shaft screw conveyer, or other direct or indirect heating device. Heating by steam injection or extrusion has the benefit of a simultaneous or sequential shearing and disintegration action which is beneficial to the hydrolysis process. The heating causes the limonene content to be decreased through evaporation and steam stripping. The limonene is then recovered by condensation of the removed steam and decanting (or centrifuging) the recovered liquid. The citrus waste solids slurry is then cooled and adjusted for pH, followed by simultaneous hydrolysis and fermentation using an enzyme mixture and fermentation organisms such as yeast, E. coli strain KO11, or other bacteria or fungi, all while being continually mixed using high solids pumps or high solids mixers. After fermentation, the ethanol is separated by distillation and the resulting residue can then be pressed and dried for use as cattle feed or further processed with fermentation using E. coli KO11, to produce more ethanol and acetate, or the unfermented galacturonic acid monomers/polymers and five carbon sugars may be recovered as additional products.

In another embodiment of the process, the ground citrus peel is first directly hydrolyzed using an enzyme mixture (the enzymes are not significantly inhibited by the limonene) with controlled pH and temperature levels to maximize simple sugar content and then the limonene content is lowered using either a decanter (or tricanter) centrifuge or filtration device to remove the solids which are high in limonene content. Recovery of limonene from the solids cake or filtrate is accomplished by solvent extraction, or alternatively by steam stripping as described in the preferred embodiment described above. The liquid solution obtained from the centrifuge or filtration process is high in sugars and low in limonene content. The solution is adjusted for pH and temperature, and fermentated using either traditional fermentation yeast, genetically engineered E. coli KO11, or other microorganisms to produce ethanol. The ethanol is separated by distillation. Following fermentation and distillation, the resulting residue may be pressed and dried for use as cattle feed with optional recovery of acetate, five carbon sugars, and galacturonic acid monomers/polymers.

The relevant prior art includes the following patent documents:

| Patent No. | | |
|---|---|---|
| (U.S. unless stated otherwise) | Inventor | Issue Date |
| 3,966,984 | Cocke, et al. | Jun. 29, 1976 |
| 4,113,573 | Gerow | Sep. 12, 1978 |
| 4,503,079 | King, et al. | Mar. 5, 1985 |
| 4,547,226 | Milch, et al. | Oct. 15, 1985 |
| 4,488,912 | Milch, et al. | Dec. 18, 1984 |
| 4,818,250 | Whitworth | Apr. 4, 1989 |
| 5,198,074 | Villavicencio, et al. | Mar. 30, 1993 |
| 4,915,707 | Whitworth | Apr. 10, 1990 |
| 4,952,504 | Pavilon | Aug. 28, 1990 |
| 5,135,861 | Pavilon | Aug. 4, 1992 |
| 6,143,337 | Fishman, et al. | Nov. 7, 2000 |
| 6,151,799 | Jones | Nov. 28, 2000 |
| 6,267,309 Bl | Chieffalo, et al. | Jul. 31, 2001 |

None of the above patents discloses a process like the present invention for yielding ethanol and other byproducts from citrus processing waste.

### SUMMARY OF THE INVENTION

The primary object of the present invention is to provide a system and method of producing ethanol from citrus processing waste.

Another object of the present invention is to increase the recovery of limonene from citrus waste in order to reduce pollution from limonene that results when raw citrus waste is converted into cattle feed by current drying processes.

A further object of the present invention is to increase the recovery of limonene from citrus waste in order be sold as a high value byproduct.

A further object of the present invention is to provide such a process that produces ethanol and byproducts for a lower cost than ethanol produced from corn.

An even further object of the present invention is to provide a process that yields other byproducts including five carbon sugars, galacturonic monomers/polymers, and a citrus based feed product for cattle and pets that has higher protein content and value than the citrus based cattle feed made from current processes.

The present invention fulfills the above and other objects by providing a system and method for producing ethanol from citrus waste that reduces limonene in the citrus peel in order that fermentation can take place to yield ethanol. This system includes means for reducing the particle size of citrus waste solids in a citrus waste slurry to a predetermined size when necessary for processing, utilizing a hammer mill, grinding pump or similar shredding/chopping/grinding apparatus.

Limonene is then removed using one of two techniques:
1. The citrus peel in the slurry is pre-hydrolyzed using a jet cooker, extruder, or other direct or indirect heating device, which pasteurizes the slurry and then it is passed through a flash tank or tube to remove the water vapor with limonene. Rapid cooling of the slurry can be achieved by vacuum cooling which gives the additional benefit of further limonene removal. However, other direct or indirect heat exchange methods can be used for cooling and stripping the slurry. pH is adjusted to suitable range for enzymes and microorganisms and hydrolysis, and potentially simultaneous fermentation, is then accomplished with enzymes with or without addition of ethanol producing microorganisms. Hydrolysis and/or fermentation in a slurry may be accomplished using an enzyme mixture circulated using high solids pumps or mixed using a high solids mixer or agitator.
2. The citrus waste is hydrolyzed using an enzyme mixture circulated using high solids pumps, or mixed using a high solids mixer or agitator, while pH and temperature are kept in a suitable range. Then the slurry can be centrifuged to remove the suspended solids which are high in limonene content, or alternatively or additionally heated by a jet cooker and a flash tube or tank used to remove limonene as in 1. above.

After the limonene has been reduced to a sufficiently low level, fermentation is accomplished in a fermentation tank using yeasts, E. coli/KO11, or other ethanol producing organisms such as fungi, E. coli, or Z. mobilis, and enzymes which may be mixed by circulation with high solids pumps or high solids mixers. Finally, ethanol can be distilled from the fermented citrus waste/beer. Optionally and additionally, the resulting residue can be further processed into solids and pet or cattle feed using a centrifuge and/or press and drying devices. Furthermore, the residue may also yield acetate, galacturonic acid monomers and polymers, and five carbon sugars.

The pH of the citrus waste is controlled throughout the process in the range of pH 1 to 13 (preferred range pH 2 to 11) by addition of acids/bases to optimize the hydrolysis by enzymes and/or cooking and to optimize fermentation outputs.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a block diagram illustrating the ethanol production process of the present invention in which limonene is removed by heating using a jet cooker and flash tube/tank prior to fermentation;

FIG. 2 is a block diagram of the ethanol production process of the present invention wherein enzymatic hydrolysis is used prior to removal of limonene using a centrifuge and/or jet cooker and flash tube or tank; and

FIG. 3 is a block diagram showing an optional sequence of fermentation with yeasts, followed by organisms capable of fermenting sugars that the yeasts fail to ferment.

### DESCRIPTION OF THE PREFERRED EMBODIMENTS

For purposes of describing the preferred embodiment, the terminology used in reference to the numbered components in the drawings is as follows:
1. Ground citrus waste slurry
2. Jet cooker
3. pH control
4. Flash tube or tank
5. Limonene
6. Vacuum tank
7. Fermentation mixing tank
8. pH control
9. High solids pump
10. Valve
11. Return line
12. Distillation
13. Ethanol
14. Centrifuge
15. Solids/cattle feed
16. Acetate, 5C sugars, GA polymers
21. Ground citrus waste slurry
22. Enzyme based hydrolysis
23. pH control
24. High solids pump
25. Valve
26. Return line
27. Centrifuge
28. Limonene
29. Fermentation tank
30. pH control
31. Distillation
32. Ethanol
33. Centrifuge
34. Solids/cattle feed
35. Acetate/GA polymers/5C sugars
41. Yeast fermentation tank
42. 1^{st} Distillation
43. Ethanol
44. Solids for cattle feed
45. Other byproducts
46. GMO fermentation tank
47. 2^{nd} Distillation
48. Ethanol
49. Other byproducts
50. Solids for cattle feed

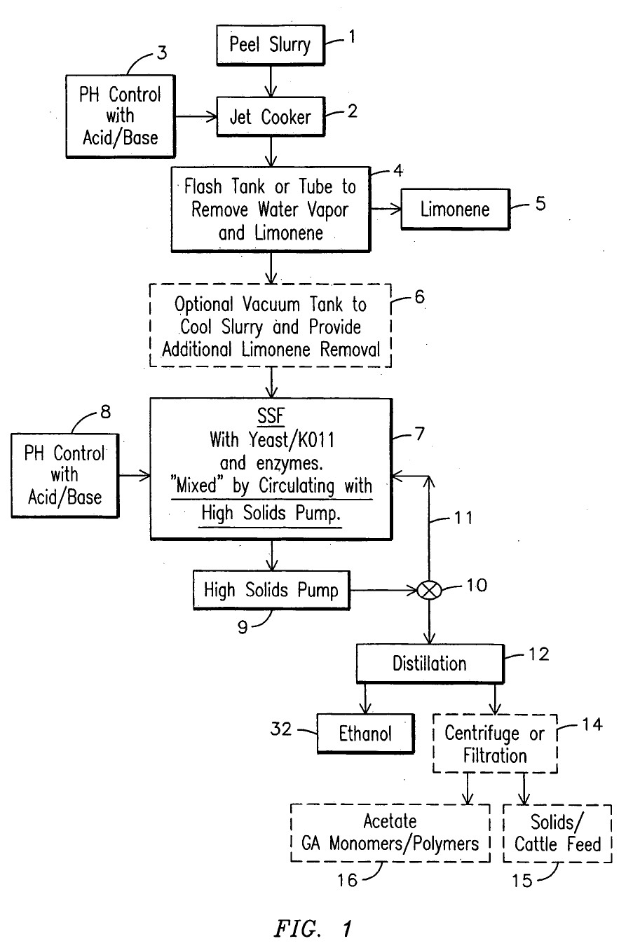

With reference to the drawings, a preferred embodiment of the ethanol production process is shown in FIG. 1 in which separation of the limonene in citrus waste is accomplished via heating and rapid cooling. FIG. 1 begins with ground citrus waste slurry 1, in which the citrus solids, consisting primarily of citrus peel, may be reduced to a pre-determined size for processing, generally less than one-half inch, by a hammer mill, grinding pump or similar shredding/chopping/grinding apparatus. The slurry pH may be adjusted to the range of pH 1 to 13 (preferred pH 2 to 11) 3 before being heated to a temperature between 60°-240° Celsius (preferred range 90°-190° C.) by steam injection 2 or, alternatively by passing through a heated hollow shaft screw conveyer or similar direct or indirect heating device. Then the slurry is injected through a venturi into a flash tube or tank 4 where the water vapor containing limonene is separated 5. The removed vapor is then condensed into a decanter and limonene run off as a liquid from the top layer. A secondary vacuum stage 6 (or other cooling device) can further reduce limonene content and rapidly cool the slurry. Next the resulting mixture is pH adjusted 8 to pH 3 to 7 and then exposed to simultaneous hydrolysis and fermentation utilizing enzymes and ethanol-producing yeasts, E. coli strain KO11, or other ethanol producing organisms such as fungi, E. coli, or Z. mobilis, in a fermentation mixing tank 7. A high solids pump 9 recirculates the mixture through a valve 10 and return line 11 until sufficient fermentation has been achieved to produce significant ethanol concentration in the mixture. As an alternative, the mixture may be mixed with a high solids mixer/auger. Once sufficient ethanol concentration is attained the mixture proceeds to distillation, or equivalent separation technology, 12 in which ethanol 13 is separated from the mixture. The residue remaining after distillation can then be processed using a centrifuge 14 or filtration device to separate the solids from liquid. Thereafter the solids can be crushed and dried for use in making a cattle feed 15. The liquid can also be further fermented using E. coli KO11 to produce additional ethanol and acetate and/or processed to produce galacturonic acid monomer/polymers or other products 16.

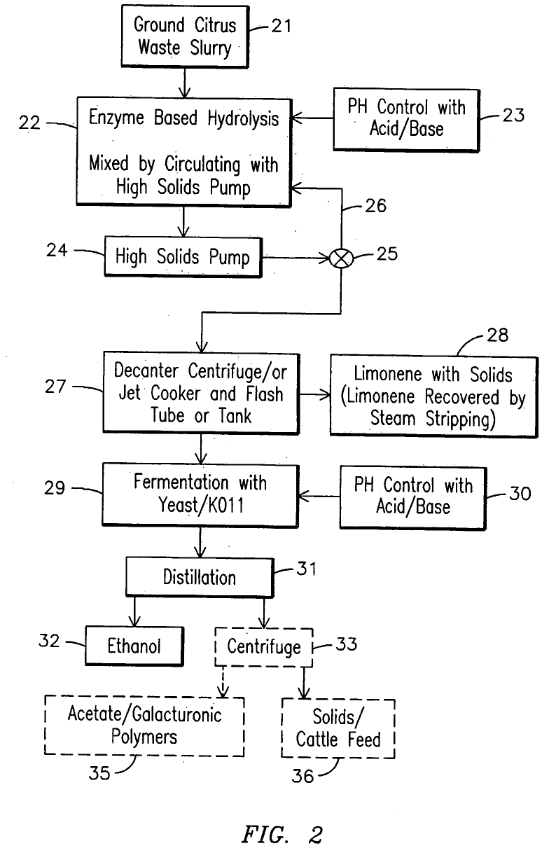

In FIG. 2 another embodiment of the ethanol production process is illustrated in which the ground citrus waste slurry is hydrolyzed using enzymes prior to limonene removal. The ground citrus waste slurry 21 is first hydrolyzed using an enzyme mixture in order to maximize simple sugar content while pH is controlled 23 in the range of pH 2 to 11, according to enzymes used. A high solids pump 24 is used to re-circulate the mixture through the valve 25 and return line 26 until sufficient hydrolysis has taken place. A centrifuge or filter device 27 is used to separate solids rich in limonene 28 from the mixture, thereby lowering the limonene content of the remaining mixture. As use of a centrifuge can be expensive, alternatively or additionally the mixture may be heated as described in FIG. 1 and then the limonene removed by condensing the steam and removing high limonene content water. Next, the mixture which now has a high sugar and low limonene content is pH controlled 30 in the range pH 3 to 7 and is then fermented in a fermentation tank 29 using either traditional fermentation yeast, E. coli KO11, or other ethanol producing organism. Distillation, or an equivalent separation technology, 31 is then used to separate the ethanol 32 from the mixture after fermentation. The residue remaining after fermentation can be exposed to a centrifuge or filtration device 33 to separate the solids, which can be pressed and dried and used as cattle feed 34. The separated liquid 35 can be used optionally to recover galacturonic acid monomers/polymers or to make additional ethanol and acetate through further fermentation using E. coli KO11 (or other organisms).

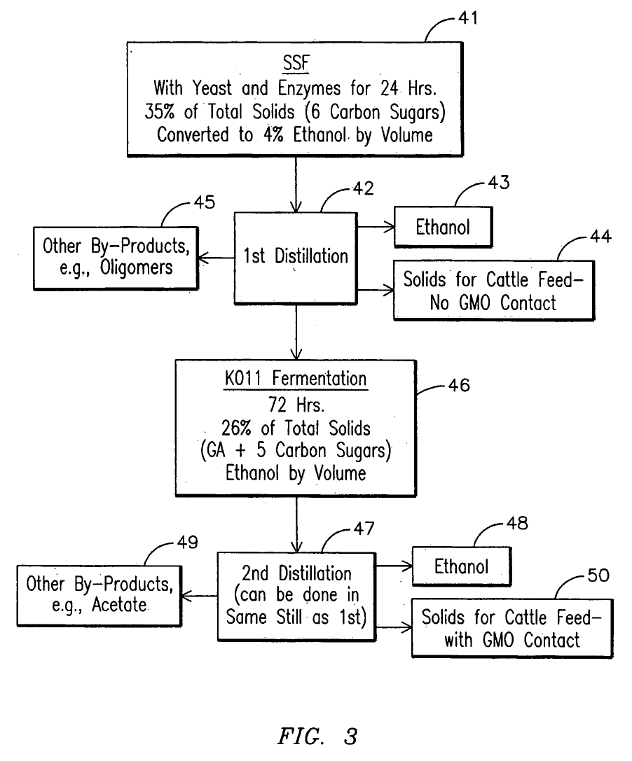

FIG. 3 shows an example of an ethanol production process of the first embodiment of the present invention where after the initial simultaneous hydrolysis and fermentation 41 (also known as simultaneous saccharification and fermentation—SSF) the ethanol 43, cattle feed 44, and other potential products 45 are recovered before the liquid rich in galacturonic acid and 5 carbon sugars that are not fermentable by unmodified yeasts undergoes a secondary fermentation 46 by E. coli strain KO11, or other ethanol producing organisms such as fungi, E. coli, or Z. mobilis. The additional ethanol 48 is then recovered along with acetate and other potential products 49, and cattle feed 50. Thus, the present invention as described and illustrated teaches a system whereby citrus processing waste can be efficiently converted into ethanol and other byproducts. Although only a few embodiments of the present invention have been described in detail hereinabove, all improvements and modifications to this invention within the scope or equivalents of the claims are included as part of this invention.

1. A system for producing ethanol from citrus waste comprising:
means for partially hydrolyzing a slurry of citrus waste to produce a citrus waste mixture containing limonene and citrus waste solids;
means for removing limonene from the citrus waste mixture; and
means for fermenting the citrus waste mixture to yield ethanol, citrus waste solid residue and remaining liquids.

2. The system of claim 1 further comprising:
means for reducing solids in citrus waste to a pre-determined particle size using one from a group of apparatuses including a hammer mill, grinding pump, shredder, chopper and grinder prior to the means for hydrolyzing the slurry of citrus waste.

3. The system of claim 1 wherein:
the means for partially hydrolyzing a slurry of citrus waste comprises heating the slurry using a jet cooker to produce a hot citrus waste mixture and with vapor having a high limonene content.

4. The system of claim 1 wherein:
the means for hydrolyzing the slurry of citrus waste comprises adding enzymes to the slurry and mixing the slurry.

5. The system of claim 4 wherein the mixing of the slurry is accomplished by circulating the slurry using a high solids pump.

6. The system of claim 4 wherein the mixing of the slurry is accomplished by circulating the slurry using a high solids mixer.

7. The system of claim 1 wherein:
the means for removing limonene comprises injecting the hot citrus waste mixture and water vapor into a flash tank connected to a condenser whereby the water vapor is condensed into an aqueous solution having a high limonene content so that said aqueous solution can be removed from the citrus waste mixture.

8. The system of claim 1 wherein:
the means for removing limonene from the citrus waste mixture comprises centrifuging the mixture to separate suspended citrus peel solids containing limonene content from the aqueous solution in order that the limonene can be removed from the mixture.

9. The system of claim 1 wherein:
the means for removing limonene from the hydrolyzed citrus waste mixture comprises filtering the mixture to separate citrus peel solids from the mixture.

10. The system of claim 1 further comprising:
means for cooling the citrus waste mixture prior to fermenting the citrus waste in order that the enzymes and fermentation agents can be added to the mixture at a suitable temperature.

11. The system of claim 10 wherein:
the means for cooling the citrus waste mixture comprises vacuum cooling.

12. The system of claim 1 wherein:
the means for fermenting the citrus waste mixture to yield ethanol comprises mixing the citrus waste mixture with at least one from a group of fermentation agents, including yeast and E. coli KO11.

13. The system of claim 1 further comprising:
means for processing the citrus waste solid residue to yield cattle feed and other byproducts.

14. The system of claim 13 wherein:
the means for processing the citrus waste solid residue into cattle feed comprises centrifuging, pressing and drying.

15. The system of claim 1 further comprising:
fermenting the remaining liquid in the mixture to yield ethanol and acetate in a two stage system where a non-GMO fermenting organism is used in the first stage and a GMO fermenting organism is used in the second stage.

16. A method for producing ethanol from citrus waste comprising the steps of:
a. hydrolyzing a slurry of ground citrus waste solids to yield a mixture of citrus waste solids in an aqueous solution;
b. removing limonene from the mixture to produce a low limonene citrus waste mixture; and
c. fermenting the low limonene citrus waste mixture to yield ethanol and other byproducts.

17. The method of claim 16 wherein:
hydrolyzing the slurry of ground citrus waste solids comprises heating the mixture to approximately 60 to 240 degrees Centigrade using a jet cooker to produce water vapor containing limonene.

18. The method of claim 16 wherein:
hydrolyzing the slurry of ground citrus waste solids comprises adding enzymes and mixing using a high solids pump while adjusting pH and temperature of the mixture.

19. The method of claim 16 wherein:
hydrolyzing the slurry of ground citrus waste solids comprises adding enzymes and mixing using a high solids mixer while adjusting pH and temperature of the mixture.

20. The method of claim 17 wherein:
removing limonene from the mixture comprises condensing the water vapor containing limonene into two immiscible liquids which can be easily removed from the mixture.

21. The method of claim 18 wherein:
removing limonene from the mixture comprises centrifuging the mixture to separate solids containing limonene from the aqueous solution.

22. The method of claim 21 further comprises:
heating the mixture to approximately 60 to 240 degrees Centigrade to produce water vapor and condensing the water vapor by temperature reduction to produce water containing limonene for easy removal from citrus waste solids.

23. The method of claim 16 further comprising a step prior to step c of:
controlling the temperature for optimal enzyme and fermentation agent performance.

24. The method of claim 16 further comprising a step prior to step c of:
controlling the pH for optimal enzyme and fermentation agent performance.

25. The method of claim 16 further wherein:
fermenting comprises initial fermentation by a non-GMO organism to recover ethanol and other products and a secondary fermentation by a GMO organism to produce additional ethanol and other products.

26. The method of claim 23 wherein the temperature is controlled using vacuum cooling.

27. The method of claim 24 wherein the pH of the mixture is controlled by adding either base or acid compounds as necessary.
